(19) United States
(12) Patent Application Publication
(10) Pub. No.: US 2016/0355858 A1
(43) Pub. Date: Dec. 8, 2016

(54) METHODS OF PROCESSING SUGAR CANE AND SWEET SORGHUM WITH INTEGRATED CONVERSION OF PRIMARY AND LIGNOCELLULOSIC SUGARS

(71) Applicants: Inbicon A/S, Fredericia (DK); Odebrecht Agroindustrial SA, Sao Paulo (BR)

(72) Inventors: Martin Dan Jeppesen, Odense V (DK); Carlos Eduardo Calmonovici, Campinas (BR); Edmar Lopes Faleiros, Campinas (BR)

(73) Assignees: Inbicon A/S, Fredericia (DK); Odebrecht Agroindustrial SA, Sao Paulo (BR)

(21) Appl. No.: 15/117,969

(22) PCT Filed: Feb. 11, 2015
(86) PCT No.: PCT/DK2015/050030

§ 371 Date: Aug. 10, 2016

Related U.S. Application Data
(60) Provisional application No. 61/938,412, filed on Feb. 11, 2014.

(51) Int. Cl. C12P 19/14; C10L 5/44; C07C 31/08; C12P 19/02; C12P 7/14

(52) U.S. Cl. CPC C12P 19/14; C12P 19/02; C12Y 302/01004; C12P 7/14; C07C 31/08; C10L 5/445; C12P 2201/00; C10L 2290/24; C10L 2290/26; C10L 2290/544; C10L 2290/547

(57) ABSTRACT

The current invention concerns methods and products related to the production of alcohol from sugar cane and/or sweet sorghum with integration of 1st and 2nd generation (1G/2G) biorefining, thus comprising the integrated conversion of primary and secondary soft lignocellulosic biomass. In particular, methods of processing sugar cane and/or sweet sorghum feedstock are disclosed, said methods comprising the steps of providing raw juice from the feedstock, recovering a residual bagasse, pretreating the bagasse and mixing it with some quantity of raw juice, and hydrolyzing the pretreated bagasse enzymatically.

### FIELD OF THE INVENTION

The present inventions relates to biotechnology, in particular to the production of alcohol, fermentable sugars and/or fermentation products in general from plant biomass, including plant primary sugars, such as fructose and sucrose, as well as from monomeric sugars derived from cellulose and other polysaccharides, such as glucose and xylan. In particular, the current invention concerns methods and products related to the production of alcohol from sugar cane and/or sweet sorghum with integration of 1^{st} and 2^{nd} generation biorefining, comprising the integrated conversion of primary and secondary lignocellulosic sugars.

### BACKGROUND OF THE INVENTION

Sugar cane juice is widely used as a source of food and fermentable sugars and, particularly in Brazil, as a source of sugars for fermentation to ethanol. While sweet sorghum is not particularly useful as a source of food sugar, it has similar potential with sugar cane as a fuel ethanol crop. Sweet sorghum is a fast-growth crop, consumes less water and fertilizer than sugar cane, and reaches peak in a different season. In some contexts, sugar cane and sweet sorghum can be optimally grown in crop rotation.

Sugar cane and sweet sorghum processing to produce ethanol is typically conducted in a similar manner: Fresh canes are pressed to produce a sugar-rich juice, also called “raw juice”, which is typically concentrated and effectively sterilized by evaporative processes, then directly fermented to ethanol. Sorghum further comprises starch-rich seeds, which are typically subject to hydrolysis using amylase and glucoamylase enzymes for instance to produce a fermentable solution. Residual lignocellulosic materials in both cases such as bagasse, straw and leaves have been used typically as a fuel for steam (heat and power) generation.

In recent years, there was considerable interest for the integrated processing schemes, whereby whole-crop sugar cane and sweet sorghum can be utilized in ethanol production. Integration requires conversion not only of primary sugars such as sucrose, glucose and fructose in pressed juice, through so-called “first generation (1G)” processes, but also conversion of cellulosic sugars, and eventually also hemicellulosic sugars, through “second generation (2G)” technologies. Lignocellulosic 2G sugars are typically obtained through a process whereby bagasse, straw and/or leaves are first pretreated and then subject to enzymatic hydrolysis using a cellulase based enzyme preparation. Because of limitations of its physical structure, lignocellulosic biomass cannot be effectively converted to fermentable sugars by enzymatic hydrolysis without some pretreatment process. A wide variety of different pretreatment schemes have been reported, each offering different advantages and disadvantages. For review see Agbor et al. (2011); Girio et al. (2010); Alvira et al. (2010); Taherzadeh and Karimi (2008). From a sustainability perspective, hydrothermal pretreatments are especially attractive. These processes utilize pressurized steam/liquid hot water at temperatures on the order of 160-230° C. to gently melt hydrophobic lignin that is intricately associated with cellulose strands, to solubilize a major part of the hemicellulose, rich in five carbon (C5) sugars, and to disrupt cellulose strands so as to improve accessibility to productive enzyme bindings. Hydrothermal pretreatments can be conveniently integrated with existing coal- and biomass-combustion electrical power generation plants to efficiently utilize turbine steam and power production capacity.

A number of schemes and process arrangements have been reported for optimizing ethanol production from sugar cane by integration of 1G and 2G processes. See e.g. (for sugar cane) Dias et al. (2013); Palacios-Bereche et al. (2013); Macrelli et al. (2012); Dias et al. (2012); Dias et al. (2011); Walter et al. (2010) and (for sorghum) Kim et al. (2012); Ceclan et al. (2012). In all such integrated processing schemes reported to date, overall conservation of process steam is considered to be a critical factor. Steam is typically used to power processes such as evaporative concentrators, ethanol distillation systems, hydrothermal pretreatment systems, and other systems.

The more process steam can be conserved, the more lignocellulosic material can be utilized for ethanol production, rather than for steam generation.

Two very recent studies considered a variety of different process configurations in optimizing integrated 1G/2G ethanol production from sugar cane, Dias et al. (2013) and Palacios-Bereche et al. (2013). One process variable considered in detail was the conditions under which enzymatic hydrolysis of pretreated bagasse was conducted. It is well known in the art that higher monomeric sugar yields can be obtained at any given enzyme dose where enzymatic hydrolysis is conducted at low total solids concentration. To the extent that the concentration of pretreated biomass total solids in the hydrolysis slurry is greater than about 5% by weight, the greater the biomass concentration, the lower will be the conversion yield at any given enzyme dose. Both Dias et al. (2013) and Palacios-Bereche et al. (2013) concluded that, despite higher yields at the enzymatic hydrolysis stage, low solids hydrolysis was disadvantageous overall because a greater quantity of steam is required to concentrate the resulting cellulosic sugar stream prior to fermentation, compared with hydrolysis at higher solids content, which results in higher sugar concentrations and reduced requirement for evaporative concentration. In alternative process scenarios, where the lignocellulosic hydrolysate is directly fermented without evaporative concentration, and without blending in to the 1G sugar process stream, higher sugar concentrations in the hydrolysate are advantageous because this, in turn, results in higher ethanol concentration in the eventual fermentation broth. Higher ethanol concentrations in fermentation broth leads to lower steam consumption in distillation (ethanol recovery). Lower steam consumption directly correlates with higher ethanol production, where lignocellulosic bagasse is alternatively used to produce steam or lignocellulosic sugars for fermentation.

### SUMMARY OF THE INVENTION

The current invention concerns methods and products related to 1G/2G integration, in particular the production of alcohol from sugar cane and/or sweet sorghum, comprising the integrated conversion of primary and secondary lignocellulosic sugars. Methods of processing sugar cane and/or sweet sorghum feedstock are disclosed, said methods comprising the steps of providing raw juice from the soft lignocellulosic biomass comprising feedstock, recovering a residual bagasse; pretreating the bagasse and mixing it with some quantity of raw juice; and hydrolyzing the pretreated bagasse enzymatically.

Thus, in a first aspect, the current invention concerns a method of processing sugar cane and/or sweet sorghum feedstock comprising the steps of:
(a) extracting raw juice from the feedstock, such as by pressing and/or crushing, and to recover a residual bagasse;
(b) pretreating the bagasse from step (a);
(c) mixing the pretreated bagasse form step (b) with some quantity of raw juice; and
(d) hydrolysing the pretreated bagasse by enzymatic hydrolysis using a cellulase enzyme preparation under conditions where the aqueous liquid phase of the hydrolysis mixture comprises at least 5, 10, 15, 20, 25, 30, 40, 50, 60 g/L sucrose derived from the added raw juice.

In some embodiments, the invention provides a method of processing sugar cane or sweet sorghum comprising the steps of
providing sugar cane or sweet sorghum raw feedstock
extracting (pressing, crushing or otherwise) raw juice from the cane feedstock so as to recover a residual bagasse
hydrothermally pretreating the bagasse
mixing the pretreated bagasse with raw juice, and
hydrolysing the pretreated bagasse by enzymatic hydrolysis using a cellulase enzyme preparation under conditions where the aqueous liquid phase of the hydrolysis mixture comprises at least 5.0 g/L sucrose derived from the added raw juice, such as at least 10, 15, 20, 25 g/L, 30 g/L, 40 g/L, 50 g/L, or 60 g/L sucrose derived from the added raw juice.

In some embodiments the pretreated bagasse may be hydrolysed using a whole slurry, comprising substantially all of the pretreated biomass both dissolved and undissolved.

In some embodiments the pretreated bagasse may be subject to a solid/liquid separation step so as to provide a fiber fraction and a liquid fraction, wherein the fiber fraction is separately subject to enzymatic hydrolysis.

A second aspect relates to one or more products, including fermentation product(s), hydrolysates, dissolved solids, mixtures or dissolved solids and raw juice, as well as intermediary products, said product(s) being obtained or obtainable by a process according to the invention, including fuel or fuel additives for generation of power, heat and/or steam.

### SHORT DESCRIPTION OF THE DRAWINGS

FIG. 1: Glucan conversion as function of time for the six shake flasks. Conditions: 170 h hydrolysis at 50° C. with 0.16 mL (10.4 FPU) AcTrio/g glucan, 12% TS*), pH 4.7-5.2 adjusted with Ca(OH)₂.

FIG. 2: Average glucan conversion after 145 h over % of added 1G sugar juice (raw juice). Conditions: 145 h hydrolysis at 50° C. with 0.16 mL (10.4 FPU) AcTrio/g glucan, 12% TS*), pH 4.7-5.2 adjusted with Ca(OH)₂. *) % TS relates to total (suspended) solids.

### DETAILED DESCRIPTION OF THE INVENTION

### Definitions

The term “dry matter (DM %)” as used herein refers to total solids (dissolved and undissolved) expressed as weight %.

“Autohydrolysis” refers to a pretreatment process wherein it is believed that acetic acid liberated by hemicellulose hydrolysis during pretreatment further catalyzes hemicellulose hydrolysis. This may apply to any hydrothermal pretreatment of lignocellulosic biomass, usually conducted at pH between 3.5 and 9.0.

As used herein the term “whole slurry” refers to an enzymatic hydrolysis reaction mixture in which the ratio by weight of undissolved to dissolved solids at the start of enzymatic hydrolysis is less than 2.2:1.

In the context of the present invention, the term “cellulase” is meant to comprise enzyme compositions that hydrolyse cellulose (beta-1, 4-D-glucan linkages) and/or derivatives thereof. Cellulases include the classification of exo-cellobiohydrolases (CBH), endoglucanases (EG) and beta-glucosidases (BG) (EC3.2.191, EC3.2.1.4 and EC3.2.1.21). Examples of cellulases include cellulases from e.g. Penicillium, Trichoderma, Humicola, Fusarium, Thermomonospora, Cellulomonas, Clostridium and Aspergillus. Suitable cellulases are commercially available and known in the art. Commercial cellulase preparations may comprise one or more further enzymatic activities. Furthermore “cellulase” can also be used interchangeably with “cell-wall modifying enzyme”, referring to any enzyme capable of hydrolysing or modifying the complex matrix polysaccharides of the plant cell wall, such as any enzyme that will have activity in the “cell wall solubilization assay” as e.g. described in WO10115754, which is herewith included by reference. Included within this definition of “cell-wall modifying enzyme” are cellulases, such as cellobiohydrolase I and cellobiohydrolase II, endoglucanases and beta-glucosidases, xyloglucanases and hemicellulolytic enzymes, such as xylanases.

Commercially available cellulase preparation(s) suitable in the present context are often optimized for lignocellulosic biomass conversion and may comprise a mixture of enzyme activities that is sufficient to provide enzymatic hydrolysis of pretreated lignocellulosic biomass, often comprising endocellulase (endoglucanase), exocellulase (exoglucanase), endoxylanase, xylosidase and B-glucosidase activities. The term “optimized for lignocellulosic biomass conversion” refers to a product development process in which enzyme mixtures have been selected and/or modified for the specific purpose of improving hydrolysis yields and/or reducing enzyme consumption in hydrolysis of pretreated lignocellulosic biomass to fermentable sugars.

In the context of the present invention, the term “glucan” is meant to comprise cellulose as well as other gluco-oligomers and other gluco-polymers. Such oligo- or polysaccharides consist of glucose monomers, linked by glycosidic bonds.

“Hydrothermal pretreatment” commonly refers to the use of water, either as hot liquid, vapor steam or pressurized steam comprising high temperature liquid or steam or both, to “cook” biomass, at temperatures of 120 degrees centigrade or higher, either with or without addition of acids or other chemicals.

“Solid/liquid separation” related terms refer to an active mechanical process, whereby liquid is separated from solid by application of force through pressing, centrifugal or other force, whereby “solid” and “liquid” fractions are provided. The separated liquid is collectively referred to as “liquid fraction.” The residual fraction comprising considerable insoluble solid content is referred to as “solid fraction.” A “solid fraction” will have a dry matter content and typically will also comprise some residual of “liquid fraction.”

“Soft lignocellulosic biomass” refers to plant biomass such as sugar cane and/or sweet sorghum according to the present invention, and relates to non-wood biomass comprising cellulose, hemicellulose and lignin.

The terms “about”, “around”, “approximately”, or “˜” indicate e.g. the measuring uncertainty commonly experienced in the art, which can be in the order of magnitude of e.g. +/−1, 2, 5, 10, 20, or even 50 percent (%), usually +/−10%.

The term “comprising” is to be interpreted as specifying the presence of the stated parts, steps, features, or components, but does not exclude the presence of one or more additional parts, steps, features, or components. E.g., a composition comprising a chemical compound may thus comprise additional chemical compounds.

In the following, the invention is disclosed in further details:

The inventors discovered that some cellulase enzyme preparations are comparatively uninhibited in an environment comprising a high percentage of raw juice from sugar cane or sweet sorghum. As a consequence, enzymatic hydrolysis using these enzyme preparations can be advantageously conducted at lower solid content where the hydrolysis mixture is supplemented with raw juice, instead of fresh water or recycled process water. The resulting hydrolysate comprises higher sugar concentration, combining both 1G and 2G sugars, and thereby permits a combined ethanol fermentation that will reach levels of ethanol in weight % that are advantageously high in terms of distillation (ethanol recovery) costs.

Table 1 shows an accounting of expected final ethanol concentration in fermentation of hydrolysate, where sugar cane bagasse has been subject to hydrothermal pretreatment and hydrolysed at various different levels of dry matter (total solids) % to equivalent conversion. Shown are values of expected ethanol in weight % where the hydrolysate is dilute using a mixture comprising 90% water, 10% cane juice, or 70% water and 30% cane juice, or 50% water and 50% cane juice. Also shown are expected ratios of enzyme consumption at the various levels of dry matter, in the absence of cane juice supplementation.

TABLE 1
Final ethanol concentration and relative enzyme consumption as a
function of DM % and % cane juice supplementation.

| | Relative | | | | |
|---|---|---|---|---|---|
| Hydrolysis | enzyme dose | Final ethanol wt % | | | |
| DM % | w/out juice | 0% juice | 10% juice | 30% juice | 50% juice |
| 18 | 1.000 | 4.63 | 4.91 | 5.47 | 6.03 |
| 17 | 0.970 | 4.37 | 4.65 | 5.21 | 5.77 |
| 16 | 0.936 | 4.11 | 4.39 | 4.95 | 5.51 |
| 15 | 0.903 | 3.85 | 4.13 | 4.69 | 5.25 |
| 14 | 0.873 | 3.60 | 3.88 | 4.44 | 5.00 |
| 13 | 0.837 | 3.34 | 3.62 | 4.18 | 4.74 |
| 12 | 0.812 | 3.08 | 3.36 | 3.92 | 4.48 |

As shown, by using a mixture of cane juice and water as diluent in hydrolysis of pretreated bagasse, equivalent final ethanol concentrations in the fermentation broth can be achieved using a substantially reduced enzyme dose. Even where cane juice imparts some inhibition of enzyme activity, it can nevertheless be advantageous to supplement hydrolysis with cane juice diluent. For example, using a commercially available cellulase preparation optimized for conversion of lignocellulosic biomass and provided by GENENCOR™ under the tradename ACCELLERASE TRIO™, supplementation of diluent in hydrolysis at 12% DM is expected to impart a loss of glucan conversion of approximately 4% in absolute yield terms, relative to hydrolysis with pure water as diluent. Yet this 4% loss in glucan conversion is readily compensated for by an approximately 16-19% savings in enzyme dose, where the hydrolysis can be run at 12% DM compared with 18% DM which would normally be required to reach final ethanol yields of 4.63 weight %, in the absence of cane juice supplementation. It is well known in the art that distillation costs are exponentially increased at ethanol concentrations beneath 4.0 weight % and fall still sharply between 4.0% and 5.0%.

The precise amount of raw juice supplementation to be used is a variable to be optimized, in light of the degree of inhibition experienced in a raw juice environment by each given cellulase enzyme preparation.

Thus, in a first aspect methods related to processing sugar cane and/or sweet sorghum feedstock are provided, wherein 1G and 2G processes are integrated. In some embodiments, a method is presented of processing sugar cane and/or sweet sorghum feedstock comprising the steps of:
(a) extracting raw juice from the feedstock, such as by pressing and/or crushing, and to recover a residual bagasse;
(b) pretreating the bagasse from step (a);
(c) mixing the pretreated bagasse form step (b) with some quantity of raw juice; and
(d) hydrolysing the pretreated bagasse by enzymatic hydrolysis using a cellulase enzyme preparation under conditions where the aqueous liquid phase of the hydrolysis mixture comprises at least 5, 10, 15, 20, 25, 30, 40, 50, or 60 g/L g/L sucrose derived from the added raw juice.

In some embodiments, the bagasse is pretreated using hydrothermal and/or autohydrolysis pretreatment.

In some embodiments, the pretreated bagasse is subject to at least one solid/liquid separation step to provide a fiber fraction and a liquid fraction; and optionally washing the fiber fraction as to remove dissolved solids, such as conducting said washing by a series of pressing and dilution steps, or other washing steps known in the art.

In some embodiments, the pretreated bagasse and/or the fiber fraction according to claim 3 is hydrolysed under conditions where initial undissolved solids are between 10 and 25%, 10 and 20%, or around 15% by weight.

In some embodiments, the pretreated bagasse and/or fiber fraction is hydrolysed under conditions where initial dissolved sucrose from the added raw juice is between 5 and 60 g/L by weight, and/or around 5, 10, 15, 20, 25, 30, 40, 50, or 60 g/L. In other embodiments, the pretreated bagasse and/or fiber fraction is hydrolysed under conditions where initial dissolved sucrose from the added raw juice is between 5 and 60 g/L, 10 and 60 g/L, 15 and 60 g/L, 20 and 60 g/L, 25 and 60 g/L, 30 and 60 g/L, 40 and 60 g/L, or 50 and 60 g/L.

In some embodiments, the pretreated bagasse and/or fiber fraction is hydrolysed under conditions where pH is maintained at pH 7.0, 6.5, 6.0, 5.5, 5.0, 4.5 or 4.0 or lower. In other embodiments, the pretreated bagasse and/or fiber fraction is hydrolysed under conditions where pH is maintained at +/−0.1-0.25 pH units around pH 7.0, 6.5, 6.0, 5.5, 5.0, 4.5 or 4.0; maintained in the range of pH 7-4, 7-5, 7-6, 6-4, 6-5, 5-4; and/or wherein the pH is maintained lower than pH 7.0, 6.5, 6.0, 5.5, 5.0, 4.5 or 4.0.

In some embodiments, the pretreated bagasse and/or fiber fraction is hydrolysed using a cellulase preparation optimized for lignocellulosic biomass conversion, such as a commercially available cellulase preparation.

In some embodiments, the pretreated bagasse and/or fiber fraction is hydrolysed using a cellulase preparation that is not inhibited more than 20% after 145 hours hydrolysis at an enzyme loading of at least 8 FPU/g DM under conditions appropriate for the tested enzyme preparation by added raw juice where sucrose derived from the added juice is at least 5 g/L. In some embodiments the pretreated bagasse and/or fiber fraction is hydrolysed using a cellulase preparation that is not inhibited more than 10, 15, 20, 25, 30, 35, 40, 45, or 50%, after 24, 48, 72, 96, 120, or 145 hours hydrolysis at an enzyme loading of at least 8 FPU/g DM under conditions appropriate for the tested enzyme preparation by added raw juice where sucrose derived from the added juice or raw juice is at least 5, 10, 15 or 20 g/L.

In some embodiments, the hydrolysate obtained after hydrolysis of pretreated bagasse and/or fiber fraction is subject to at least one solid/liquid separation step to provide insoluble solids separated from dissolved solids, such as by using a filter press with internal wash, optionally comprising a further drying step.

In some embodiments, the insoluble solids are suitable as a fuel and/or fuel additive, such as fuel and/or fuel additive for generation of power, heat and/or steam. This may require one or more conventional processing steps, such as drying and/or pelleting.

In some embodiments, the dissolved solids comprising cellulosic sugars and sugars derived from cane or sorghum juice are mixed with a further quantity of raw juice, optionally followed by a concentration step, such as evaporative concentration and/or reverse osmosis concentration. Other conventional concentration steps or procedures may be used as well. In some embodiments, the further quantity of raw juice added can e.g. be in the range of around 1, 2, 5, 10, 15, 20, 30, 40, or 50% by weight or volume; and/or at least 1, 2, 5, 10, 15, 20, 30, 40, or 50% by weight or volume.

In some embodiments, the hydrolysate obtained according to one of the above described methods is subsequently fermented to provide one or more product(s), optionally followed and/or preceded by a concentration, purification or any other step(s).

In some embodiments, the dissolved solids are subsequently fermented to provide one or more product(s), optionally followed and/or preceded by a concentration, purification or any other step(s).

In some embodiments, the mixture of dissolved solids and raw juice is subsequently fermented to provide one or more product(s), optionally followed and/or preceded by a concentration, purification or any other step(s).

In some embodiments any combination of hydrolysate, dissolved solids, including mixture of dissolved solids and raw juice, as well as any concentrated solution subsequently fermented to provide one or more product(s), optionally followed and/or preceded by a concentration, purification or any other step(s).

Commonly, fermentation, especially fermentation in the field of ethanol production is performed using yeast, often Saccharomyces, such as Saccharomyces cerevisiae. Alternatives are known in the art, especially when aiming at provision of other fermentation products than ethanol.

Thus, according to some embodiments, one or more fermentation products are provided being e.g. one or more chemical, alcohol, ethanol or any combination thereof.

Generally, hydrolysis can be performed in different ways. According to some embodiments, hydrolysis is either performed as whole slurry. According to other embodiments, a solid/liquid separation step is performed prior to hydrolysis so as to provide a fiber fraction and a liquid fraction, wherein the fiber fraction is separately subject to enzymatic hydrolysis.

Further embodiments relate to one or more products comprising or consisting essentially of the hydrolysate, the dissolved solids, the mixture of dissolved solids and raw juice, and any concentrate provided as described herein. This includes also any combination of any hydrolysate, dissolved solids, mixtures of dissolved solids and raw juice, and any concentrates.

Further product related embodiments pertain to a fuel or fuel additive, such as fuel and/or fuel additive for generation of power, heat and/or steam. These can be provided from the insoluble solids separated from dissolved solids, such as by using a filter press with internal wash, optionally comprising a further drying step as described herein. Such fuel or fuel additives are believed of power, heat and/or steam. In some embodiments, the fuel is provided as solid fuel, such as in the form of pellets.

Some embodiments relate to further products, comprising 0.1-99.9% weight/weight or volume/volume of any product according to the present invention.

### NUMBERED EMBODIMENTS

In the following, further embodiments of invention are presented in the following lists of numbered embodiments (A and B):

### Numbered Embodiments A

1a. A method of processing sugar cane or sweet sorghum comprising the steps of
extracting (pressing, crushing or otherwise) raw juice from the cane feedstock so as to recover a residual bagasse
hydrothermally pretreating the bagasse
mixing the pretreated bagasse with some quantity of raw juice, and
hydrolysing the pretreated bagasse by enzymatic hydrolysis using a cellulase enzyme preparation under conditions where the aqueous liquid phase of the hydrolysis mixture

comprises at least 5, 10, 15, 20, 25, 30, 40, 50 or 60 g/L sucrose derived from the added raw juice.

2a. The method of embodiment 1a wherein bagasse is pretreated using hydrothermal 1 pretreatment.

3a. The method of embodiment 2a wherein bagasse is pretreated using autohydrolysis hydrothermal pretreatment.

4a. The method of embodiment 1a wherein pretreated bagasse is subject to at least one solid/liquid separation step to provide a fiber fraction and a liquid fraction.

5a. The method of embodiment 4a wherein the fiber fraction is washed so as to remove dissolved solids.

6a. The method of embodiment 5a wherein washing is conducted by a series of pressing and dilution steps.

7a. The method of embodiment 4a wherein pretreated fiber fraction is hydrolysed under conditions where initial undissolved solids is between 10 and 25% by weight.

8a. The method of embodiment 4a wherein pretreated fiber fraction is hydrolysed under conditions where initial dissolved sucrose from added cane or sorghum juice is between 5 and 60 g/L by weight.

9a. The method of embodiment 1a wherein pretreated bagasse is hydrolysed under conditions where initial undissolved solids is between 10 and 25% by weight.

10a. The method of embodiment 1a wherein pretreated bagasse is hydrolysed under conditions where initial dissolved sucrose from added cane or sorghum juice is between 5 and 60 g/L.

11a. The method of embodiment 1a wherein pretreated bagasse is hydrolysed under conditions where pH is maintained at 4.5 or lower.

12a. The method of embodiment 1a wherein pretreated bagasse is hydrolysed using a commercially available cellulase preparation optimized for lignocellulosic biomass conversion.

13a. The method of embodiment 1a wherein pretreated bagasse is hydrolysed using a cellulase preparation that is not inhibited more than 20% after 145 hours hydrolysis at an enzyme loading of at least 8 FPU/g DM under conditions appropriate for the tested enzyme preparation by added cane juice where sucrose derived from the added juice is at least 5 g/L.

14a. The method of embodiment 1a wherein hydrolysate obtained after hydrolysis of pretreated bagasse is subject to at least one solid/liquid separation step to provide insoluble solids separated from dissolved solids.

15a. The method of embodiment 14a wherein the solid/liquid separation step is performed using a filter press with internal wash.

16a. The method of embodiment 14a wherein insoluble solids are used as a solid fuel for steam generation.

17a. The method of embodiment 14a wherein dissolved solids comprising cellulosic sugars and sugars derived from cane or sorghum juice are mixed with cane or sorghum juice prior to a concentration step.

18a. The method of embodiment 17a wherein the concentration step is performed using evaporative concentration.

19a. The method of embodiment 17a wherein the concentration step is performed using reverse osmosis concentration.

20a. The method of embodiment 14a wherein dissolved solids comprising cellulosic sugars and sugars derived from cane or sorghum juice are subject to a concentration step.

21a. The method of embodiment 14a wherein the concentrated solution is subsequently fermented to produce ethanol, optionally after intermediate concentration, purification or other steps.

22a. The method of embodiment 17a wherein the concentrated solution is subsequently fermented to produce ethanol, optionally after intermediate concentration, purification or other steps.

23a. The method of embodiment 14a wherein the concentrated solution is subsequently fermented to produce chemicals, optionally after intermediate concentration, purification or other steps.

24a. The method of embodiment 17a wherein the concentrated solution is subsequently fermented to produce chemicals, optionally after intermediate concentration, purification or other steps.

### Numbered Embodiments B

1b. A method of processing sugar cane and/or sweet sorghum feedstock comprising the steps of: (a) extracting raw juice from the feedstock, such as by pressing and/or crushing, and to recover a residual bagasse; (b) pretreating the bagasse from step (a); (c) mixing the pretreated bagasse from step (b) with some quantity of raw juice; and (d) hydrolysing the pretreated bagasse from step (c) by enzymatic hydrolysis using a cellulase enzyme preparation under conditions where the aqueous liquid phase of the hydrolysis mixture comprises at least 5, 10, 15, 20, 25, 30, 40, 50, or 60 g/L sucrose derived from the added raw juice.

2b. The method of embodiment 1b, wherein bagasse is pretreated using hydrothermal and/or autohydrolysis pretreatment.

3b. The method according to embodiment 1b or 2b, wherein pretreated bagasse is subject to at least one solid/liquid separation step to provide a fiber fraction and a liquid fraction; and optionally washing the fiber fraction as to remove dissolved solids, such as conducting said washing by a series of pressing and dilution steps.

4b. The method according to any one of the preceding embodiments B, wherein the pretreated bagasse and/or the fiber fraction according to embodiment 3 is hydrolysed under conditions where initial undissolved solids are between 10 and 25%, 10 and 20%, or around 15% around by weight.

5b. The method according to any one of the preceding embodiments, wherein the pretreated bagasse and/or fiber fraction is hydrolysed under conditions where initial dissolved sucrose from the added raw juice is between 5 and 60 g/L, 10 and 60 g/L, 15 and 60 g/L, 20 and 60 g/L, 25 and 60 g/L, 30 and 60 g/L, 40 and 60 g/L, or 50 and 60 g/L; and/or around 5, 10, 15, 20, 25, 30, 40, 50, or 60 g/L.

6b. The method according to any one of the preceding embodiments B, wherein the pretreated bagasse and/or fiber fraction is hydrolysed under conditions where pH is maintained around pH 7.0, 6.5, 6.0, 5.5, 5.0, 4.5 or 4.0, such as maintained at +/−0.1-0.25 pH units around pH 7.0, 6.5, 6.0, 5.5, 5.0, 4.5 or 4.0; maintained in the range of pH 7-4, 7-5, 7-6, 6-4, 6-5, 5-4; and/or wherein the pH is maintained lower than pH 7.0, 6.5, 6.0, 5.5, 5.0, 4.5 or 4.0.

7b. The method according to any one of the preceding embodiments B, wherein the pretreated bagasse and/or fiber fraction is hydrolysed using a cellulase preparation optimized for lignocellulosic biomass conversion, such as a commercially available cellulase preparation.

8b. The method according to any one of the preceding embodiments B, wherein the pretreated bagasse and/or fiber fraction is hydrolysed using a cellulase preparation that is not inhibited more than 10, 15, 20, 25, 30, 35, 40, 45, or 50%, after 24, 48, 72, 96, 120, or 145 hours hydrolysis at an enzyme loading of at least 8 FPU/g DM under conditions appropriate for the tested enzyme preparation by added raw juice where sucrose derived from the added juice or raw juice is at least 5, 10, 15 or 20 g/L. All combinations/permutations of % inhibition, hours of hydrolysis and g/L sucrose from the (raw) juice are herewith disclosed specifically.

9b. The method according to any one of the preceding embodiments B, wherein the hydrolysate obtained after hydrolysis of pretreated bagasse and/or fiber fraction is subject to at least one solid/liquid separation step to provide insoluble solids separated from dissolved solids, such as by using a filter press with internal wash, optionally comprising a further drying step.

10b. The method according to embodiment 9b, wherein insoluble solids are suitable as a fuel and/or fuel additive, such as fuel and/or fuel additive for generation of power, heat and/or steam.

11b. The method according to embodiments 9b or 10b, wherein the dissolved solids comprising cellulosic sugars and sugars derived from cane or sorghum juice are mixed with a further quantity of raw juice, optionally followed by a concentration step, such as evaporative concentration and/or reverse osmosis concentration, wherein the further quantity of raw juice added can e.g. be in the range of around 1, 2, 5, 10, 15, 20, 30, 40, or 50% by weight or volume.

12b. The method according to any one of the preceding embodiments B, wherein (i) the hydrolysate obtained according to any one of the preceding embodiments; (ii) the dissolved solids obtained according to embodiment 9b; (iii) the mixture of dissolved solids and raw juice obtained according to embodiment 11b; (iv) the concentrated solution provided through the concentration step according to embodiment 11b; and/or or any combination of (i), (ii), (iii) and/or (iv) is subsequently fermented to provide one or more product(s), optionally followed and/or preceded by a concentration, purification or any other step(s).

13b. The method according to embodiment 12b, wherein the fermentation product is one or more chemical, alcohol, ethanol or any combination thereof.

14b. The method according to any one of the preceding embodiments, wherein the hydrolysis is either performed as whole slurry, comprising substantially all of the pretreated biomass both dissolved and undissolved, or wherein a solid/liquid separation step is performed prior to hydrolysis so as to provide a fiber fraction and a liquid fraction, wherein the fiber fraction is separately subject to enzymatic hydrolysis.

15b. A fermentation product provided according to any one of the preceding embodiments B.

16b. A product comprising or consisting essentially of the hydrolysate provided according to any one of the preceding embodiments B; the dissolved solids provided according to embodiment 9b; the mixture of dissolved solids and raw juice provided according to embodiment 11b; the concentrated solution provided through the concentration step according to embodiment 11b; and/or or any combination of (i), (ii), (iii) and/or (iv).

17b. A fuel or fuel additive, such as fuel and/or fuel additive for generation of power, heat and/or steam provided according to embodiment 9b or 10b.

18b. A further product, comprising 0.1-99.9% weight/weight or volume/volume one or more of the product according to embodiment 16b or 17b.

### EXAMPLES

### Example 1

### Characterization of Raw Juice from Sugar Cane and Sweet Sorghum

Juice from sugar cane and sweet sorghum can be characterized to determine sugars composition.

Cane juice was extracted by pressing to provide juice, then irradiated using X-ray irradiation to eliminate contaminating microorganisms, then stored at 4° C. until use. Typical concentration(s)/composition(s) of sugar cane juice are in the range of 13 to 16 Brix, with around the following sugar composition: total sugar=saccharose (97%)+fructose (1.5%)+glucose (1.5%).

Composition of sugar cane juice soluble dry substance have also been published to be:

| | g/100 g |
|---|---|
| Juice constituent | |
| Sugars | 75.0-94.0 |
| Sucrose | 70.0-90.0 |
| Glucose | 2.0-4.0 |
| Fructose | 2.0-4.0 |
| Oligosaccharides | 0.001-0.050 |
| Salts | 3.0-4.5 |
| of inorganic acids | 1.5-4.5 |
| of organic acids | 1.0-3.0 |
| Organic acids | 1.5-5.5 |
| Carboxilic acids | 1.1-3.0 |
| Amino acids | 0.5-2.5 |
| Other organic non-sugars | |
| Protein | 0.5-0.6 |
| Starch | 0.001-0.18 |
| Soluble polysaccharides | 0.03-0.50 |
| Waxes, fats, phosphatides | 0.04-0.15 |

(Source: Sugar Technology, Beet and Cane Sugar Manufacture (1998) Van der Poel, P. W.; Schiweck, H.; Schwartz, T; see e.g. Table 2/21, pag. 153)

### Example 1

### Characterization of Selected Cellulase Enzyme Preparations

A cellulase preparation can be obtained from Trichoderma reesei RUT-C30 raised on C5-rich liquid fraction from pretreated sorghum bagasse as carbon source, as described by Korpos et al. (2012).

A cellulase preparation can be obtained from Penicillium echinulatum raised on pretreated sugar cane bagasse as carbon source, as described by Pereira et al. (2013). A cellulase preparation can be obtained from Aspergillus sp. S₄B₂F raised on wheat bran as carbon source, as described by Soni et al. (2010)

A cellulase preparation from Trichoderma harzianum raised on pretreated sugar cane bagasse as carbon source, as described by Delabona et al. (2012).

A cellulase preparation from Bacillus subtilis KIBGE HAS raised on sugar cane bagasse as carbon source, as described by Bano et al. (2013).

A commercially available cellulase preparation optimized for conversion of lignocellulosic biomass and sold by NOVOZYMES™ under the tradename CELLIC CTEC3™ can be obtained commercially.

A commercially available cellulase preparation optimized for conversion of lignocellulosic biomass and sold by GENENCOR™ under the tradename ACCELLERASE TRIO™ can be obtained commercially.

A commercially available cellulase preparation optimized for conversion of lignocellulosic biomass and sold by DSM™ can be obtained commercially.

A commercially available cellulase preparation optimized for conversion of lignocellulosic biomass and sold by Dyadic™ can be obtained commercially.

The cellulase activity of the cellulase preparations can be determined and expressed per unit volume or mass as “filter paper units” as determined by the method of Adney, B. and Baker, J., Laboratory Analytical Procedure #006, “Measurement of cellulase activity”, Aug. 12, 1996, the USA National Renewable Energy Laboratory (NREL), which is expressly incorporated by reference herein in entirety. It will be readily understood by those skilled in the art that FPU provides a measure of cellulase activity, but additional enzyme activities may be usefully included in an effective mixture of cellulytic enzymes, including but not limited to hemicellulase enzyme activities.

Further examples of cellulase preparations can e.g. be found herein in the “Defintions”, as well as e.g. in WO10115754.

### Example 3

### Comparative Performance of Enzymatic Activity of Selected Cellulase Preparations in a Raw Juice Environment

Any of the enzyme preparations mentioned in example 2 can be used for comparative performance measurements in the presence of various amounts of cane juice, as described in Example 4

### Example 4

### Optimization of Raw Juice Supplementation for a Given Cellulase Preparation

For any given enzyme preparation, the raw juice supplementation which will be advantageous will be that which at which the final ethanol concentration in fermentation broth with added juice is equivalent to “base case” conditions, but at which the DM % of hydrolysis is sufficiently lowered so as to provide better conversion at a given enzyme dose overall, notwithstanding some inhibition of conversion imposed by the added juice. A commercially available cellulase preparation optimized for conversion of lignocellulosic biomass and sold by GENENCOR™ under the tradename ACCELLERASE TRIO™ was examined.

Cellulase activity measurements in Filter Paper Units (FPU) were determined for ACCELLERASE TRIO™ by the method of Ghose (1987) and found to be 65 FPU/g enzyme preparation. An enzyme dose of 0.16 ml/g glucan (or 10.4 FPU/g glucan) was used in the experiments.

A set of six shake flasks was set up with double determination of the three conditions: 100 wt-% 1G sugar juice, 50 wt-% 1G sugar juice and 0% sugar juice (pure water) as reference. Shake flasks were incubated with agitation at 250 rpm and 50° C. Bagasse obtained after extraction of cane juice as described in example 1 was pretreated in the Inbicon 100 kg/h pilot plant with a feed flow of 50 kg TS/h, as described by Petersen et al. (2009). Before pretreatment, the fresh bagasse (SCB batch E) was soaked in water to achieve a dry matter content of 40 wt-% TS_{105° C.} at ambient temperature without addition of any chemicals. Pretreatment conditions were 195° C., residence time 12 minutes, log severity Ro 3.88. After leaving the pretreatment reactor the pretreated biomass slurry was pressed to a fibre fraction of approximately 55% DM and a liquid fraction. An adjustment period of 3 h before steady state was kept and samples were taken. The pretreated material, fibre fraction as well as liquid fraction, was collected and analysed. The dry matter and composition of the samples were determined.

Pretreated bagasse fibre fraction obtained as described was used in shake flask experiments at a dry matter content 12% without any additives other than AcTRIO and pH adjustment chemicals. The pH was adjusted with 20% Ca(OH)₂ to pH 5. Before enzyme addition the sugar content was measured by HPLC. When preparing the sample for the HPLC the solution was diluted with sulphuric acid, whereby the sucrose is split into glucose and fructose. To follow the hydrolysis, samples were measured on HPLC after 6, 24, 50, 72, 145 and 170 hours. From the measured glucose and xylose concentrations the values measured before enzyme addition were subtracted to eliminate the contribution from 1G sugar.

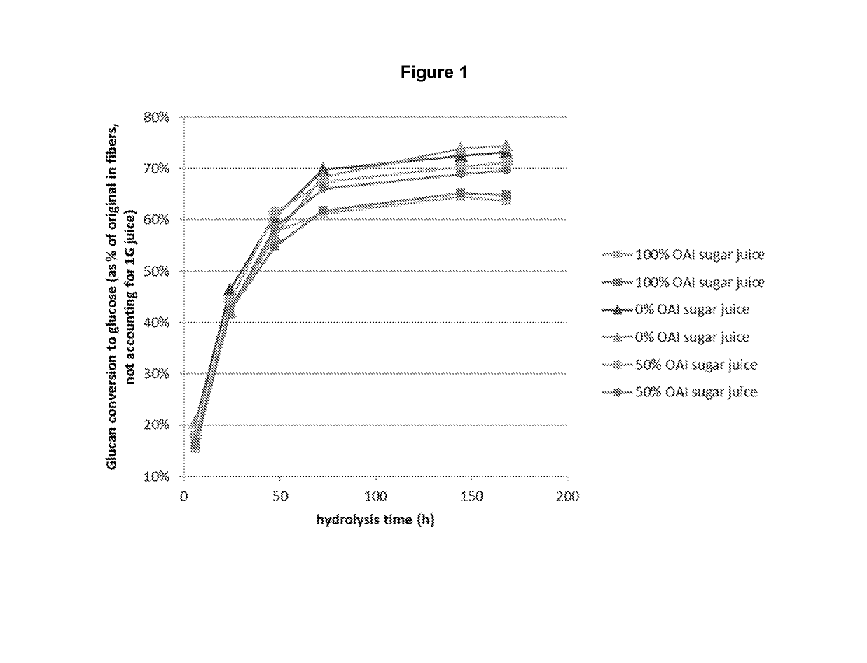

The glucan conversion over time was calculated based on the sugars from fibre fraction, the sugar from 1G juice having been subtracted. FIG. 1 shows glucan conversion for the six shake flasks. The obtained glucan conversions after 170 h for the shake flasks without use of 1G sugar juice (0% OAI sugar juice), with 50% 1G sugar juice and with 100% 1G sugar juice were determined to be approx. 73%, 70% and 65%, respectively.

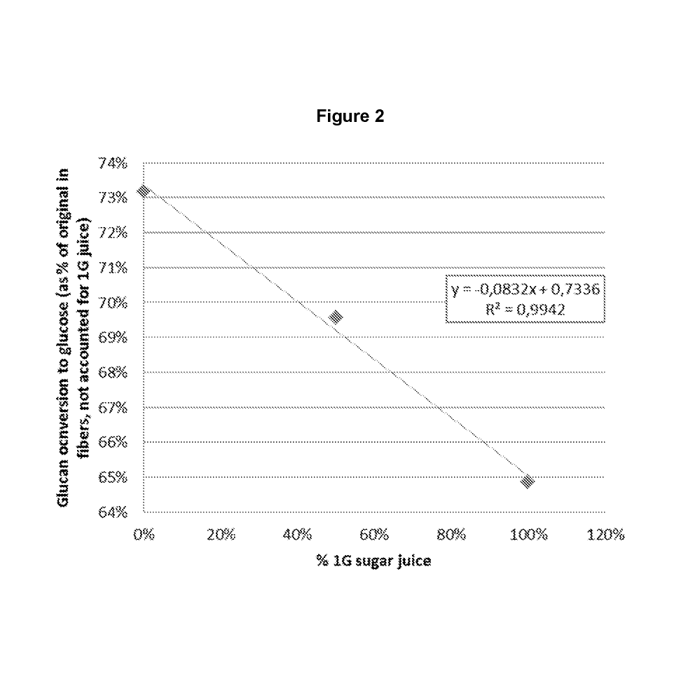

FIG. 2 shows the average glucan conversion after 145 h hydrolysis over the percentage of 1G sugar juice added to hydrolysis. This relation can be described by a linear function and shows a decrease by 8% conversion (absolute) when going from 0% sugar juice to 100% sugar juice. It is assumed that a similar or slightly lower decrease would be obtained for higher dry matter contents.

As shown, compared with a “base case” hydrolysis of pretreated bagasse as whole slurry at 18% DM, in light of Table 1, and the results presented in this example, use of cane juice supplementation in enzymatic hydrolysis of separated fiber fraction using the enzyme preparation ACCELLERASE TRIO™ could be particularly advantageous where about 30% of diluent used in hydrolysis is cane juice, with 70% water. Under these conditions, equivalent final ethanol concentrations can be achieved in fermentation broth, but hydrolysis can be conducted at 15% DM. A reduction in glucan conversion is expected corresponding to 71% vs 73% (71/73=0.973). However, at the same time, an increased conversion yield is expected at equivalent enzyme dose, where the relative enzyme equivalence dose is only 0.903. Accordingly, the conversion yield at equivalent dose is expected to be on the order of 1/0.903=1.107. A total net gain in conversion yield where hydrolysis is conducted at 15% DM using diluent comprising 30% cane juice is expected at equivalent enzyme dose of (1.107)*(0.973)=1.08, that is, approximately 5% absolute conversion increase at equivalent enzyme dose.

It will be readily understood by one skilled in the art that a similar optimization procedure can be applied to any particular enzyme preparation, based on the results.

In this case, where diluent used to dilute fiber fraction was 30% cane juice, and where hydrolysis was conducted at 15% DM, the sucrose concentration in the hydrolysate at the start of enzymatic hydrolysis comprised at least 12 g/L, assuming a sucrose concentration of at least 70 g/L in the cane juice.

It will be readily understood by a person skilled in the art that the embodiments and examples provided are descriptive, only, and not intended to limit the scope of the inventions as defined by the claims.

### REFERENCES

Agbor, V., et al. “Biomass pretreatment: Fundamentals toward application”, Biotechnology Advances (2011) 29:675
Alvira, P., et al. “Pretreatment technologies for an efficient bioethanol production process based on enzymatic hydrolysis: A review”, Bioresource Technology (2010) 101:4851
Girio, F., et al., “Hemicelluloses for fuel ethanol: A review,” Bioresource Technology (2010), 101:4775
Taherzadeh, M., et al. “Pretreatment of Lignocellulosic Wastes to Improve Ethanol and Biogas Production: A Review” International Journal Molecular Science (2008) 9:1621 Ceclan R. E., Pop A. and Ceclan M., (2012), Studies concerning the integrated use of sweet sorghum for bioethanol production in Romania, Chemical Engineering Transactions, 29, 877-882
Korpos, M. et al., (2012) Processing sweet sorghum into bioethanol—an integrated approach PERIODICA POLYTECHNICA-CHEMICAL ENGINEERING, 56(1):21 Dias, M., et al., (2013), Evaluation of process configurations for second generation integrated with first generation bioethanol production from sugarcane, Fuel Processing Technology 109:84.
Palacios-Bereche, R., et al., (2013) Exergetic analysis of the integrated first- and second-generation ethanol production from sugarcane, http://dx.doi.org/10.1016/j.energy.2013.05.010
Macrelli, S., et al., (2012) Techno-economic evaluation of 2d generation bioethanol production from sugar cane bagasse and leaves integrated with the sugar-based ethanol process, Biotechnology for Biofuel 5:22
Dias, M., et al., (2012) Improving second generation ethanol production through optimisation of first generation production process from sugarcane, Energy 43:246
Dias, M., et al., (2011) Simulation of integrated first and second generation bioethanol production from sugarcane: comparison between different biomass pretreatment methods, J. Ind. Microbiol. Biotechnol. 38:955
Walter, A., and Ensinas, A., (2010) Combined production of second-generation biofuels and electricity from sugarcane residues, Energy 35:874
Kim, M., et al., (2012) Utilization of whole sweet sorghum containing juice, leaves and bagasse for bio-ethanol production, Food Sci. Biotechnol. 21(4):1075
Pereira, B., et al., (2013) Cellulase on-site production from sugar cane bagasse using Penicillium echinulatum, Bioenerg. Res. 6:1052
Petersen, M., et al. (2009) Optimization of hydrothermal pretreatment of wheat straw for production of bioethanol at low water consumption without addition of chemicals, Biomass and Bioenergy 33:834.
Soni, S., et al., (2010) Bioconversion of sugarcane bagasse into second generation bioethanol after enzymatic hydrolysis with in-house produced cellulases from Aspergillus sp. S4B2F, BioResources 5(2):741
Bano, S., et al., (2013) High production of cellulose degrading endo-1,4,B-D-glucanase using bagasse as a substrate from Bacillus subtilis KIBGE HAS, Carbohydrate Polymers 91:300

1. A method of processing sugar cane and/or sweet sorghum feedstock comprising the steps of:
(a) extracting raw juice from the feedstock, such as by pressing and/or crushing, and to recover a residual bagasse;
(b) pretreating the bagasse from step (a);
(c) mixing the pretreated bagasse from step (b) with some quantity of raw juice; and
(d) hydrolysing the pretreated bagasse from step (c) by enzymatic hydrolysis using a cellulase enzyme preparation under conditions where the aqueous liquid phase of the hydrolysis mixture comprises at least 5, 10, 15, 20, 25, 30, 40, 50, or 60 g/L g/L sucrose derived from the added raw juice.

2. The method of claim 1, wherein bagasse is pretreated using hydrothermal and/or autohydrolysis pretreatment.

3. The method according to claim 1 or 2, wherein pretreated bagasse is subject to at least one solid/liquid separation step to provide a fiber fraction and a liquid fraction; and optionally washing the fiber fraction as to remove dissolved solids, such as conducting said washing by a series of pressing and dilution steps.

4. The method according to any one of the preceding claims, wherein the pretreated bagasse and/or the fiber fraction according to claim 3 is hydrolysed under conditions where initial undissolved solids are between 10 and 25%, 10 and 20%, or around 15% by weight.

5. The method according to any one of the preceding claims, wherein the pretreated bagasse and/or fiber fraction is hydrolysed under conditions where initial dissolved sucrose from the added raw juice is between 5 and 60 g/L by weight, and/or around 5, 10, 15, 20, 25, 30, 40, 50, or 60 g/L.

6. The method according to any one of the preceding claims, wherein the pretreated bagasse and/or fiber fraction is hydrolysed under conditions where pH is maintained at pH 7.0, 6.5, 6.0, 5.5, 5.0, 4.5 or 4.0 or lower.

7. The method according to any one of the preceding claims, wherein the pretreated bagasse and/or fiber fraction is hydrolysed using a cellulase preparation optimized for lignocellulosic biomass conversion, such as a commercially available cellulase preparation.

8. The method according to any one of the preceding claims, wherein the pretreated bagasse and/or fiber fraction is hydrolysed using a cellulase preparation that is not inhibited more than 20% after 145 hours hydrolysis at an enzyme loading of at least 8 FPU/g DM under conditions appropriate for the tested enzyme preparation by added raw juice where sucrose derived from the added juice is at least 5 g/L.

9. The method according to any one of the preceding claims, wherein the hydrolysate obtained after hydrolysis of pretreated bagasse and/or fiber fraction is subject to at least one solid/liquid separation step to provide insoluble solids separated from dissolved solids, such as by using a filter press with internal wash, optionally comprising a further step such as a drying step.

10. The method according to claim 9, wherein insoluble solids are suitable as a fuel and/or fuel additive, such as fuel and/or fuel additive for generation of power, heat and/or steam.

11. The method according to claim 9 or 10, wherein the dissolved solids comprising cellulosic sugars and sugars derived from cane or sorghum juice are mixed with a further quantity of raw juice, optionally followed by a concentration step, such as evaporative concentration and/or reverse osmosis concentration.

12. The method according to any one of the preceding claims, wherein (i) the hydrolysate obtained according to any one of the preceding claims; (ii) the dissolved solids obtained according to claim 9; (iii) the mixture of dissolved solids and raw juice obtained according to claim 11; (iv) the concentrated solution provided through the concentration step according to claim 11; and/or any combination of (i), (ii), (iii) and/or (iv) is subsequently fermented to provide one or more product(s), optionally after concentration, purification or any other step(s).

13. The method according to claim 12, wherein the fermentation product is one or more chemical, alcohol, ethanol or any combination thereof.

14. The method according to any one of the preceding claims, wherein the hydrolysis is either performed as whole slurry, or wherein a solid/liquid separation step is performed prior to hydrolysis so as to provide a fiber fraction and a liquid fraction, wherein the fiber fraction is separately subject to enzymatic hydrolysis.

15. A fermentation product provided according to any one of the preceding claims.

16. A product comprising or consisting essentially of the hydrolysate provided according to any one of the preceding claims; the dissolved solids provided according to claim 9; the mixture of dissolved solids and raw juice provided according to claim 11; the concentrated solution provided through the concentration step according to claim 11; and/or or any combination of (i), (ii), (iii) and/or (iv).

17. A fuel or fuel additive, such as fuel and/or fuel additive for generation of power, heat and/or steam provided according to claim 10.

18. A further product, comprising 0.1-99.9% weight/weight or volume/volume one or more of the product according to claim 16 or 17.
